(19) United States
(12) Patent Application Publication
(10) Pub. No.: US 2024/0358734 A1
(43) Pub. Date: Oct. 31, 2024

(54) DISCOVERY OF NOVEL TRPV1 CHANNEL BLOCKERS BY HIGH-THROUGHPUT SCREENING OF MARINE NATURAL PRODUCTS

(71) Applicants: The Hong Kong University of Science and Technology, Hong Kong (CN); Southern Marine Science and Engineering Guangdong Laboratory (Guangzhou), Guangzhou City (CN)

(72) Inventors: Peiyuan QIAN, Hong Kong (CN); Pingbo HUANG, Hong Kong (CN); Wenkang YE, Hong Kong (CN); Sin Tung LUI, Hong Kong (CN); Qirui ZHAO, Hong Kong (CN); Aifang CHENG, Hong Kong (CN)

(21) Appl. No.: 18/647,744

(22) Filed: Apr. 26, 2024

Related U.S. Application Data
(60) Provisional application No. 63/498,526, filed on Apr. 27, 2023.

(51) Int. Cl. A61K 31/7048; A61K 31/55; A61K 47/20; A61P 29/00

(52) U.S. Cl. CPC A61K 31/7048; A61K 31/55; A61K 47/20; A61P 29/00

(57) ABSTRACT

The subject invention pertains to hexacyclic xanthones isolated from marine bacteria. These chrexanthomycins are effective TRPV1 inhibitors that are effective as analgesics.

### CROSS-REFERENCE TO RELATED APPLICATION

This application claims the benefit of U.S. Provisional Application Ser. No. 63/498,526, filed Apr. 27, 2023, which is hereby incorporated by reference in its entirety including any tables, figures, or drawings.

### BACKGROUND OF THE INVENTION

Pain is a protective reflex of the body, but it can severely impact an individual's quality of life. Statistics show that approximately 60% of people over 65, 70% of patients with cancer, and 95% of patients with spinal cord injuries suffer from chronic pain syndrome. Existing analgesics, including opioids, nonsteroidal anti-inflammatory drugs (NSAIDs), alcohol, and cannabis, have apparent side effects. For instance, opioids may lead to addiction, tolerance, respiratory depression, and seizures, while NSAIDs cause gastrointestinal bleeding and increase the risk of cardiovascular events. Therefore, the discovery of safe, potent analgesic agents with minimal side effects is a pressing unmet medical need.

### BRIEF SUMMARY OF THE INVENTION

Embodiments are directed to hexacyclic xanthones isolated from marine bacteria for use as an analgesic. The hexacyclic xanthone is selected from chrexanthomycin A (cA), chrexanthomycin B (cB), chrexanthomycin C (cC), chrexanthomycin F (cF) or any combination thereof. The efficacy is comparable to that of Capsazepine. The analgesic can be at a concentration of about 0.75 to about 20 μM in a solution. The solvent can include, but is not limited to dimethylsulfoxide (DMSO).

Embodiments are directed to a method of applying the analgesic comprising the hexacyclic xanthone in a vehicle to a site of pain in a patient. The vehicle can be a solution and can be applied by injection, topical administration, or by any other means.

### BRIEF DESCRIPTION OF THE DRAWINGS

FIG. 1 shows the chemical structures of chrexanthomycins according to embodiments.

FIG. 2A shows a schematic for analysing FM4-64 dye-uptake by TRPV1 using Zeiss Celldiscoverer 7.

FIG. 2B illustrates steps of analysing a capsaicin-induced TRPV1 activation by a dye-uptake assay.

FIG. 3A shows a bar graph comparison of the TRPV1 inhibitory performance of Chemical structures of chrexanthomycins as indicated by n2/n1 ratios.

FIG. 3B shows a bar chart of the n2/n1 ratios that indicated the inhibitory effect on TRPV1 of cC and cF relative to capsazepine using a cell-based model.

FIG. 4 shows a dose-response curve of % inhibition vs concentration for cC upper and cF lower.

FIG. 5A shows a representative trace of cC that inhibits the TRPV1 channel where patches with 10 nm of capsaicin activation are followed by 20 μM of drug application while holding potentials are +60 mV where the period of activation by capsaicin and period where the drug cC is applied to the patch and where the expansions I, II, and III indicate 3 second expansions of a baseline, capsaicin activated and drug inhibited periods.

FIG. 5B shows a representative trace of cF that inhibits the TRPV1 channel where patches with 10 nm of capsaicin activation are followed by 20 μM of drug application while holding potentials are +40 mV where the period of activation by capsaicin and period where the drug cF is applied to the patch and where the expansions I, II, and III indicate 3 second expansions of a baseline, capsaicin activated and drug inhibited periods.

FIG. 6A shows changes in relative macroscopic current before and after the addition of cC, Left and cF Right.

FIG. 6B shows a bar chart that compares changes of relative macroscopic current between capsaicin-activated state (Before) and after addition of cF or cC.

FIG. 6C shows a bar chart that indicates the mouses licking of a capsaicin-/TRPV1-induced pain sensation site treated with Capsazepine, cC and cF.

### DETAILED DISCLOSURE OF THE INVENTION

Embodiments are directed to novel hexacyclic xanthones that have strong pain inhibitory effects, with IC₅₀ values ranging from 0.75 M to 2.1 M and no apparent cellular toxicities. These xanthones demonstrate an efficacy like capsazepine in blocking capsaicin-induced pain sensation in a mouse model. Compared to known TRPV1 modulators, the distinctive pharmacophores of these novel hexacyclic xanthones suggest novel binding modes between the ligands and ion channels. These new antagonists can be employed as innovative analgesics.

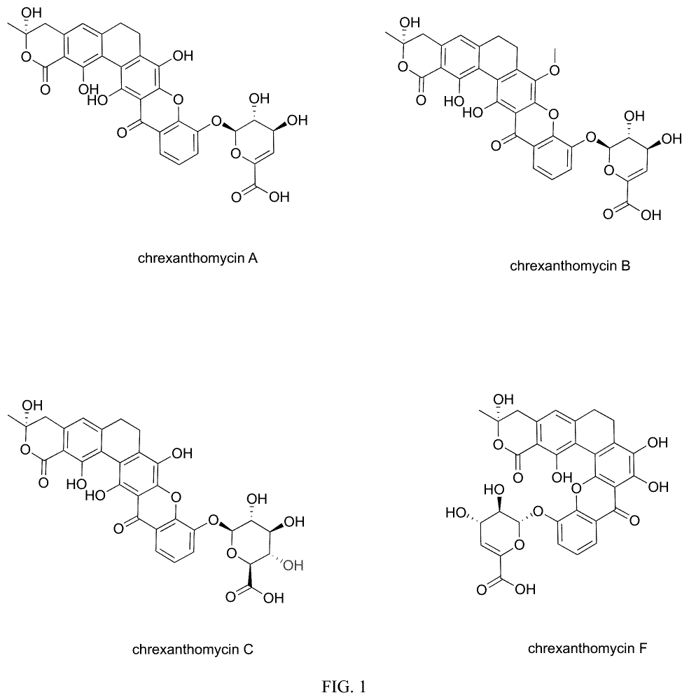

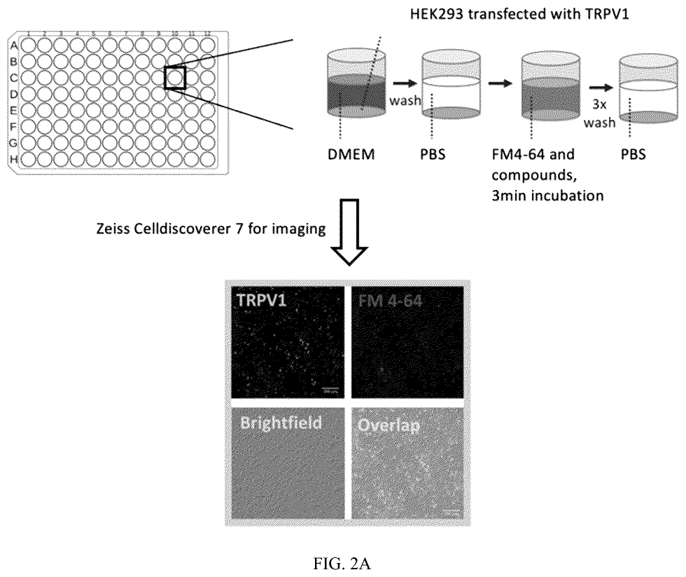

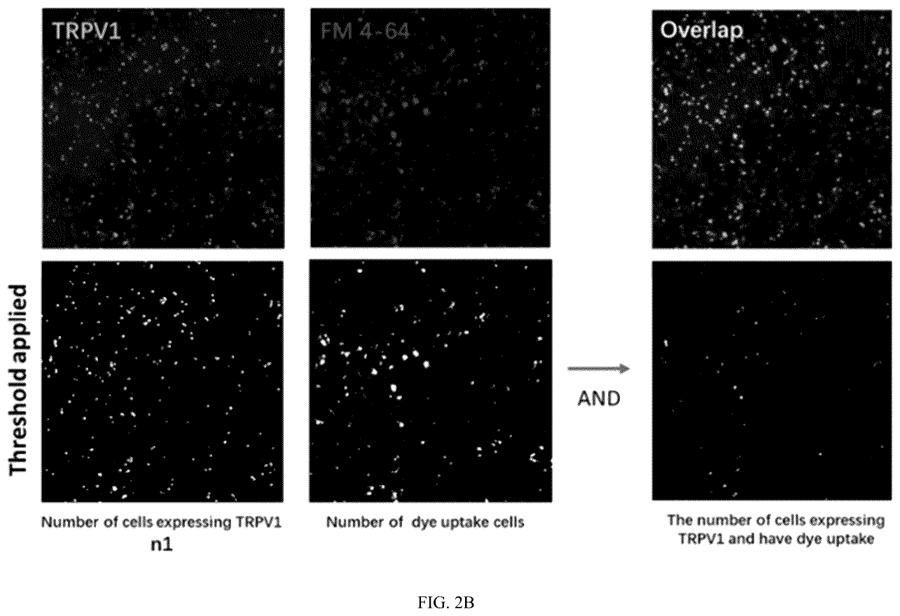

The hexacyclic xanthones, chrexanthomycins, specifically chrexanthomycin A, B, C, and F (cA, cB, cC, cF), shown in FIG. 1, demonstrate inhibitory effects on TRPV1 were discovered upon screening potential TRPV1 modulators. These compounds were identified during screening of marine bacterial metabolites. To screen potential modulators of TRPV1, a high-throughput screening system based on FM4-64 dye uptake was established using HEK293 cells transfected with hTRPV1-pIRES-eGFP and cultured in 96-well plates. The cells are incubation with 5 μM FM4-64 dye and 100 μg/ml test compounds for 3 minutes, washed, imaged using Zeiss Celldiscoverer 7, and analyzed using Image J (FIG. 2A). The number of GFP positive cells which is an indicator of the number of TRPV1 expressed cells, is calculated as n1 and the number of cells which are both GFP positive and dye uptake positive is calculated as n2 (FIG. 2B). The ratio (n2/n1) is a functional indicator of TRPV1 channel activity.

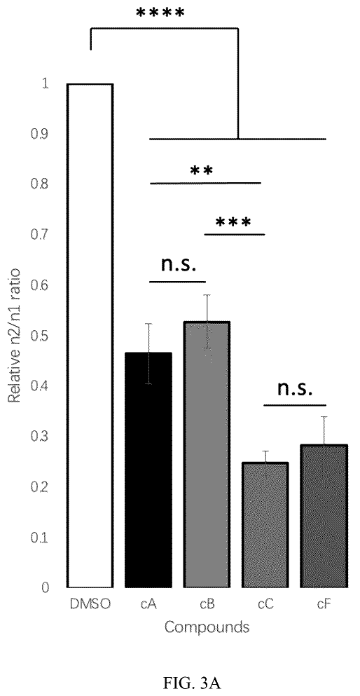

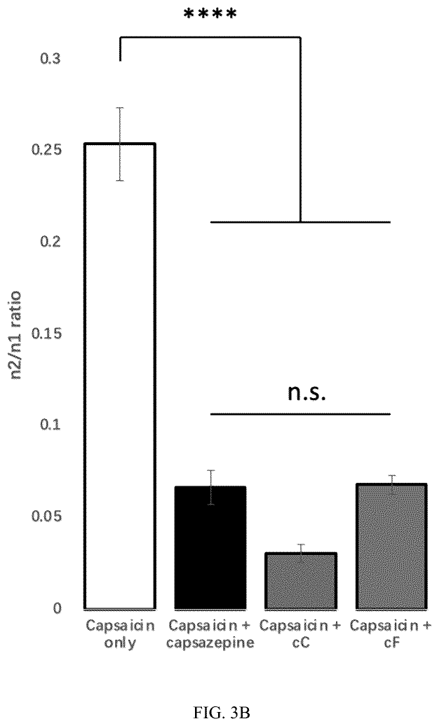

As can be seen in FIG. 1, cA, cB, and cF contain a 4′,5′-anhydroglucuronic acid moiety and a hemiketal group at ring A of the hexacyclic xanthone core while the six-membered ring system of cF bends in a different orientation than cA and cB. All four members of the chrexanthomycins possess an inhibitory effect on TRPV1, with significant decreases in the relative n2/n1 ratio compared to the vehicle control (DMSO). Among these four chrexanthomycins, cC with glucuronic acid moiety exhibited the lowest relative n2/n1 ratio (FIG. 3A) compared with its dehydration product, cA, or other chrexanthomycins with anhydroglucuronic acid moieties, suggesting the additional hydroxyl group may benefit the inhibitory effect and make cC the most effective TRPV1 inhibitor.

The performance of cC and cF with capsaicin and capsazepine, with HEK293 cells transfected with human TRPV1 and cultured on a 96-well plate are compared by the n2/n1 ratio of the groups incubated with cC and cF to those incubated with capsaicin alone and capsazepine (FIG. 3B). The mean n2/n1 ratio for the capsaicin group is 0.254, while the cC, cF, and capsazepine groups have ratios of 0.0307, 0.068, and 0.0667, respectively. ANOVA with post hoc testing revealed that the n2/n1 ratio of the cC, cF and capsazepine groups significantly differed from that of the capsaicin group. No significant difference was observed between the capsazepine group and the drug groups. Additionally, the n2/n1 ratio of the cC group is slightly lower than that of the capsazepine group, but this difference is not significant in the ANOVA test. However, a t-test shows a significant difference between cC and capsazepine (p=0.00937). This demonstrates that cC is a TRPV1 inhibitor that can outperform capsazepine in the cell-based model.

### Half Maximal Inhibitory Concentration (IC₅₀) of cC in a Cell-Based Model

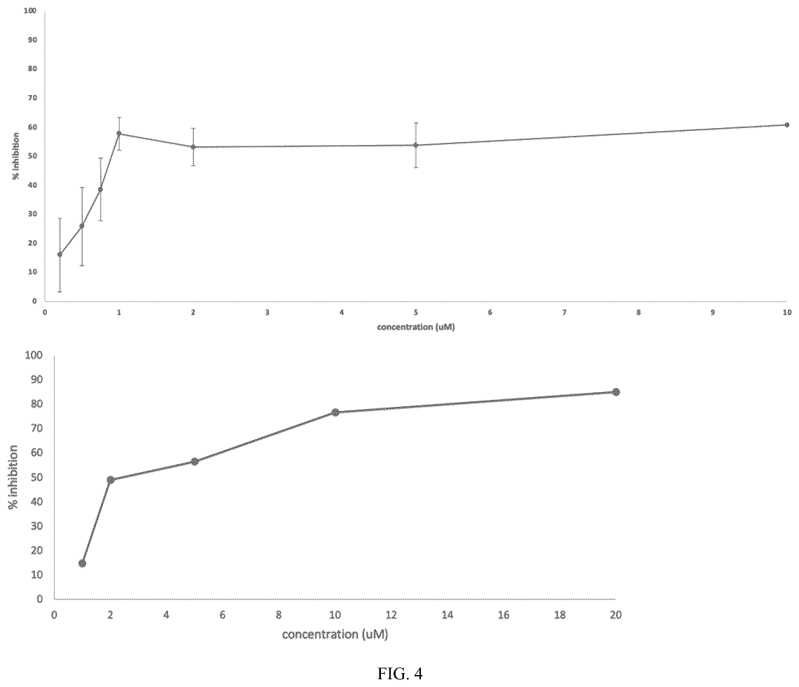

A cell-based model allows quantitative examination of the potency of cC in inhibiting the TRPV1 channel in vitro. The assay involves mixing 1 μM of capsaicin with varying concentrations of cC and 5 μM of FM4-64, and the mixture is added to each well for a 3-minute incubation. The results, normalized with a DMSO-only control (without capsaicin), of different concentrations of cC are compared with the cC-free group, which indicated fully activated TRPV1 channels and set as 100% activation. Eight different cC concentrations, including 0.2 μM, 0.5 μM, 0.75 μM, 1 μM, 2 μM, 5 μM, 10 μM, and 20 μM, plotted with a dose-response curve demonstrates a flattening trend at high concentrations (10 μM and 20 PM) of cC, where the high concentration tested once, and the inhibitory effect of cC at relatively low concentrations (less than or equal to 5 μM) curve is presented in FIG. 4.

FIG. 4 shows that the IC₅₀ of cC is within the range of 0.75 μM to 1 μM in the cell-based system and the graph indicates that cC performs best at the concentration range of 0.75 to 1 μM. However, at concentrations greater than 1 μM, the inhibitory effect of cC slightly reduces before increasing again with further concentration. For the IC₅₀ of cF, it does not reach 50% TRPV1 inhibition in the group of 2 μM, which lies within a higher range than cC.

### Chrexanthomycin C Effects on TRPV1 Channel in Patch-Clamp Assays

A cell-based high-throughput drug screening method identifies potential TRPV1 inhibitors using the FM4-64 dye-uptake assay though the assay cannot conclusively distinguish the specific mechanism of blockage as numerous factors, such as other channels, pinocytosis, and drug-induced membrane potential alteration via pore-forming, can contribute to changes in FM4-64 dye uptake. A high-resolution patch clamp assays using an excised inside-out recording configuration allows investigation of the potential inhibitory effect of cC and cF on TRPV1, where changes in the patch-clamp macroscopic current before and after drug application indicates drug effectiveness.

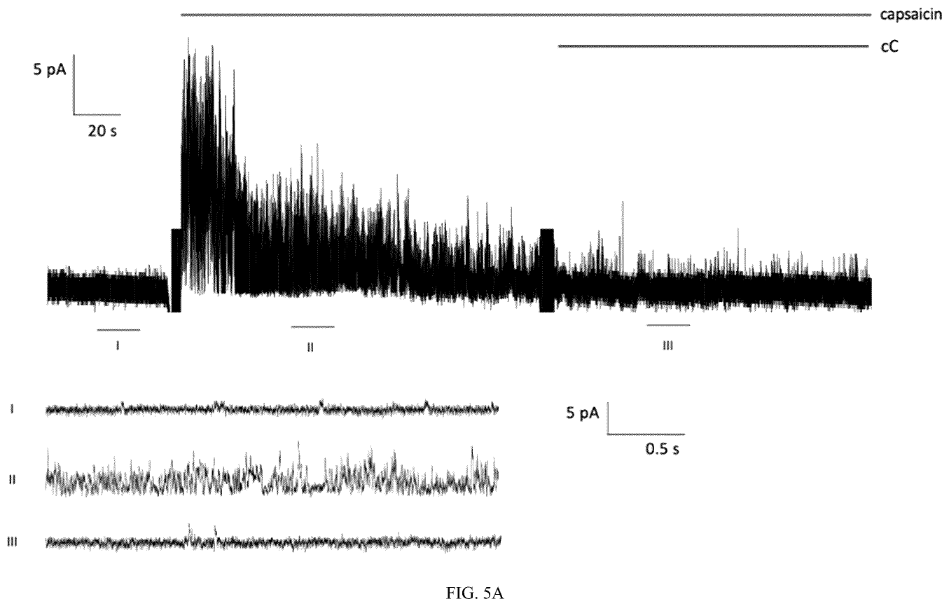

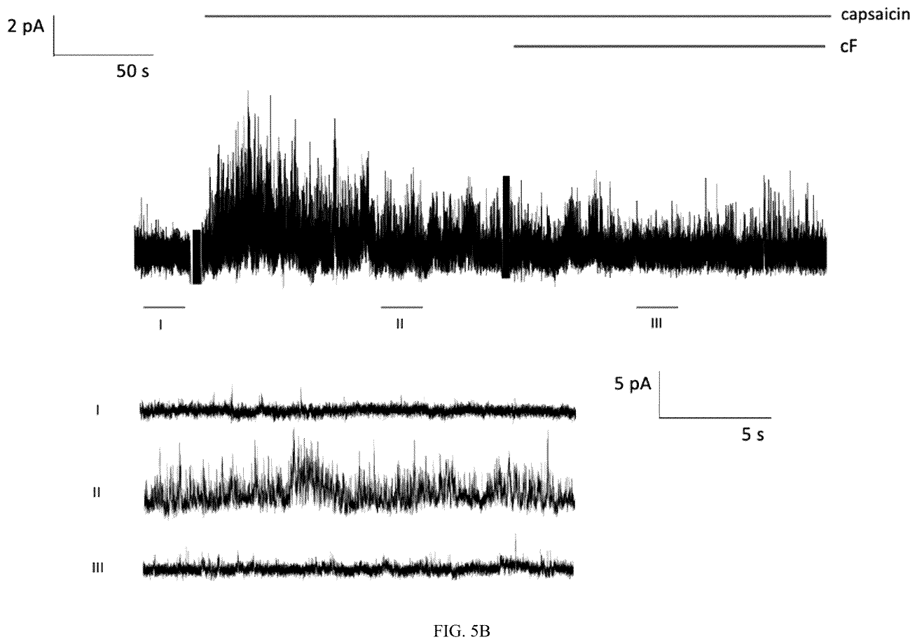

Most compounds that show inhibition in the high-throughput dye uptake screening also demonstrate a similar effect in a patch clamp study, which indicates that cC and cF effectively inhibit TRPV1 channels in the patch-clamp macroscopic current, with cC having a better inhibition than cF (FIGS. 5A and 5B), consistent with the dye uptake assay. Capsaicin-activated TRPV1-expressing patches shows a drop in macroscopic current after applying candidate drugs, suggesting inhibition of capsaicin-activated TRPV1 channels. To confirm that the inhibitory effect is specific to the TRPV1 channel, the recorded trace expanded to 3 seconds demonstrates a typical trace pattern of TRPV1 channel activation. This suggest that the TRPV1 channel specifically mediates changes after adding capsaicin and the drug.

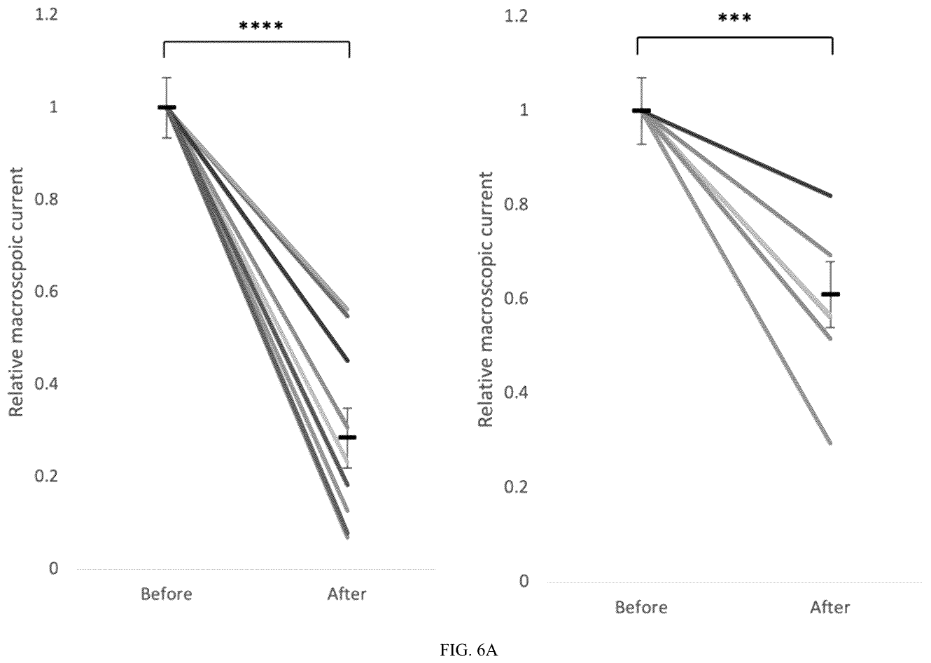

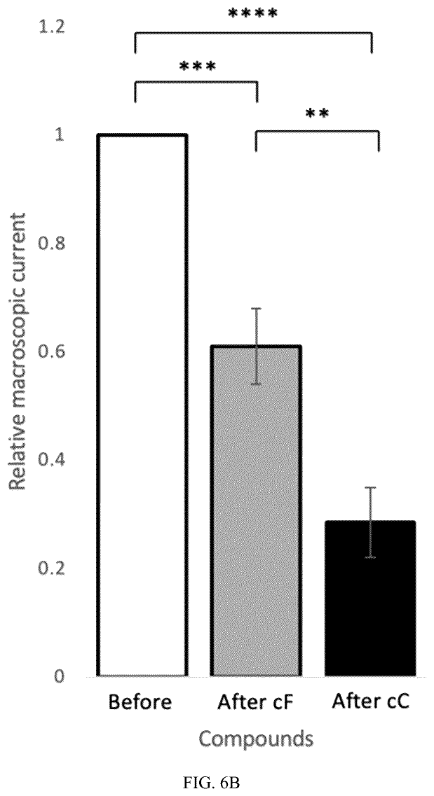

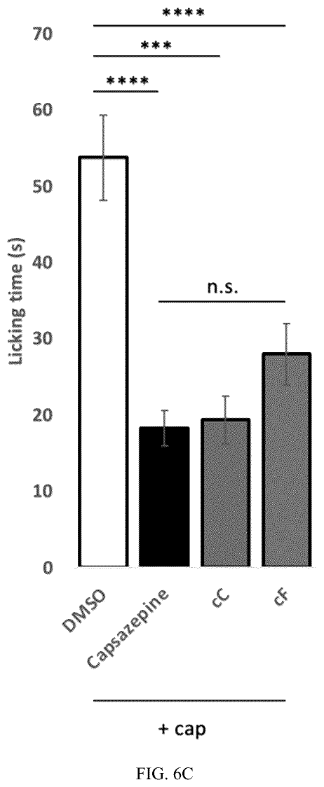

FIG. 6A shows a composite of the macroscopic currents for individual patches. Specifically, demonstrating that the macroscopic current exhibits a decreasing trend after adding cC and cF, with the cC application reducing the current more significant. In the fully capsaicin-activated state of TRPV1 channels, the average relative macroscopic current decreases to 0.285 after the cC addition. After conducting a t-test, we found a significant difference between capsaicin and cC in the drop of relative macroscopic current (p=6.980*10⁻⁹) (FIG. 6B). Taken together, the results indicate that cC exhibits a TRPV1 inhibitory effect during the capsaicin-activated state.

### Blocking Capsaicin-Induced Pain Blocking in a Mouse Model

The inhibitory effect of cC on TRPV1 demonstrated in vitro using dye uptake assay and patch clamp electrophysiology. An in vivo, behavioural experiment had drugs mixed with capsaicin injected into the right hind paws of eight one-month-old wild-type female mice. The mixture analysed using HPLC and confirms that capsaicin does not react with cC. The total time spent paw-licking, a typical nociception behaviour in mice, was recorded as an indicator of TRPV1 activation and compared with a positive control group injected with capsaicin alone. The potency of the injected drugs is related to the reduction of licking time. Another group, the negative control group, of mice was injected with a mixture of capsaicin and capsazepine to compare the inhibitory effect of cC and cF.

The mice were observed for one month after injection to check for any undesired side effects, and no observable health issues were found, suggesting the safety of both drugs. The mean licking time for the capsaicin alone group was over 50 seconds, while the mean licking time for cC and capsazepine groups were 19 and 18 seconds, respectively. ANOVA with post hoc test revealed a significant difference between the licking time of capsaicin alone and the groups injected with cC and capsazepine. In contrast, no significant difference was found between cC and capsazepine (FIG. 6C), suggesting that cC is as effective as capsazepine in blocking capsaicin-induced pain sensation.

Following are examples that illustrate procedures for practicing the invention. These examples should not be construed as limiting. All percentages are by weight and all solvent mixture proportions are by volume unless otherwise noted.

### MATERIALS AND METHODS

### Strains

Strains were purchased from the Thailand Bioresource Research Center and the Marine Culture Collection of China or isolated from the coastal water of the Red Sea.

### Fermentation and Extraction

The strain was cultured in 125-ml Erlenmeyer flasks containing 50 ml of different cultural media, AM4 (soybean powder 20 g/L, peptone bacteriological 2 g/L, glucose 20 g/L, soluble starch 5 g/L, yeast extract 2 g/L, NaCl 4 g/L, K₂HPO₄ 0.5 g/L, MgSO4.7H2O 0.5 g/L, CaCO₃ 2 g/L, 3% sea salt and set pH to 7.8), GYM (yeast extract 4 g/L, malt extract 10 g/L, glucose 4 g/L, 3% sea salt and set pH to 7.2-7.4) and AM6 (soluble starch 20 g/L, glucose 10 g/L, peptone bacteriological 5 g/L, yeast extract 5 g/L, CaCO₃ 5 g/L, 3% sea salt and set pH to 7.2-7.5) and 10-15 glass beads (3 mm in diameter) at 2 different temperatures-room temperature and 30° C., and with an agitation of 180 rpm for 1 week. The bacterial culture broth was extracted with an equal volume of ethyl acetate three times to obtain the crude extract. Crude extract of S. chrestomyceticus BCC 24770 cultured in AM5 media and under 30° C. exhibited significant modulation activities and cultured on a large scale to accumulate enough.

### Isolation and Purification

Around 60 g of crude extract was obtained from 30 L culture broth and was separated by reverse-phase C18 column chromatography and eluted with 20, 40, 60, 80, and 100% acetonitrile/water to obtain different fractions. Bioactive TRPV1 modulators were rich in the moderate polarity fractions. 40% fraction was further purified by semi-preparative HPLC (Waters 2695 Separations Module; Milford, USA) and eluted with an isocratic mobile phase at a flow rate of 3 ml min⁻¹. (Solution A: acetonitrile; solution B: Milli-Q water. Acetonitrile concentration at 33% and flushed for 50 min. The eluate was monitored at a UV wavelength of 230 nm (Waters 2998 Photodiode Array Detector). Compounds were collected, nitrogen-blow dried, and dissolved in dimethyl sulfoxide (DMSO) for further biological assessments. Semi-preparative HPLC was performed using a C18 column 250×10 mm in size and designed for a particle size of 5 m.

### Structural Elucidation

¹H NMR was performed on the 500 and 800 MHz Varian spectrometers, and ¹³C NMR spectra were obtained on the 200 MHz Varian spectrometers. Standard 2D NMR experimental spectra, including HSQC, HMBC, and COSY, were collected at 25° C. MS data were recorded from the Bruker ultrafleXtreme ultrahigh-resolution TOF LC-MS system. HRMS m/z=621.1235 [M+Na]⁺(calcd for C₃₁H₂₅O₁₄, 621.1239, 6=−0.64 ppm).

### Reagents and Plasmids

Capsaicin and capsazepine were from Sigma-Aldrich. The expression vector hTRPV1-IRES-EGFP was generated by subcloning human TRPV1 cDNA into the selected competent cell; Top10 was used due to its high transformation efficiency. Heat shock the mixture of Top10 cells with hTRPV1-IRES-EGFP and evenly spread the mixture on the Kanamycin dish, and incubate for 12-18 hours. After incubation, the colony was formed and expanded by transferring a selected colony into the LB medium. The mixture was cultured on a shaker for another 12-18 hours. After colony expansion, the plasmids were extracted with TIANprep Midi Plasmid Kit (TIANGEN).

### Cell Culture and Transfection

HEK293T (RRID: CVCL_1926) cells were obtained from ATCC, and these cells were assumedly authenticated by ATCC and were not further authenticated. The cell line was routinely tested negative for mycoplasma contamination and maintained in Dulbecco's Modified Eagle Medium (DMEM) supplemented with 10% fetal bovine serum and 100 U/mL penicillin/streptomycin (Life Technologies) in an atmosphere of 95% air 5% CO₂ at 37° C. Cell transfection was performed using the PEI (3 μL/g plasmid).

### High-Throughput FM4-64 Dye-Uptake Assays and Imaging

Transfected HEK293 cells in a 96-well plate were divided into 8 groups, each containing 12 wells. 10-11 wells of each group were set as experimental groups in which cells were incubated with compound mixtures. 1-2 wells of each group were set as control groups in which cells were incubated with control solution A or B. One group was operated simultaneously to reduce the difference between wells. Cells in DMEM were washed with CaCl₂-HBSS buffer once to remove the remnant medium. Subsequently, cells were incubated with the compound mixture or control solution for 2-3 min at RT. After FM4-64 incubation, cells were washed with CaCl₂-HBSS buffer thrice to remove FM4-64 residuals. A group of cells was subjected to Zeiss Celldiscoverer 7 for fluorescent imaging.

To separate GFP and FM4-64 fluorescent dye, we used an LED light source at 470 nm to excite the GFP channel and light at 567 nm to excite the FM4-64 channel. 5× objective (Plan Apo, dry, autocorrect, NA 0.35), Orca camera (Orca Flash 4.0 v3), CO2 Module S1 incubation system, and Zenblue software were used for image recording. Each image comprises a GFP channel, FM4-64 channel, and bright field channel. And 4 non-overlapping images were taken for each well using the Tile function at Zenblue.

### Image Analysis for Drug Screening

All images were analysed with ImageJ software. The same threshold would be applied to all the images in the same experiment group to select GFP-positive and dye uptake-positive cells. Cell number was calculated by the “Analyze particle” function in ImageJ. GFP-positive and dye uptake-positive cells were selected using the “AND” function in the image calculator. The number of GFP-positive cells (TRPV1 expressed cells) would be calculated as N1, and the number of cells that are both GFP positive and dye uptake positive would be calculated as N2. The ratio of N1/N2 represents the percentage of TRPV1 expressed cells that uptake FM4-64 fluorescent dye and would be used to analyse TRPV1's function.

### Patch-Clamp Electrophysiological Studies

3T3 cells were recorded using excised inside-out voltage clamp. Patch electrodes with a resistance of 4 MΩ±1.1 MΩ were pulled on a glass pipette puller (DMZ Zeitz-Puller). 3T3 cells were first transferred to a patch container to minimize the use of drugs and transfected with hTRPV1-IRES-EGFP. Immediately before assay, the cell medium was changed to PBS as well as the pipette medium. The dish was connected to the ground electrode via the salt bridge. Glass pipette filled with PBS was inserted into the patch electrode. A positive pressure of around 40 mmHg was applied to the pipette and soaked into the cell medium. The offset was tuned to near 0 pA when the holding potential was 0 mV. Pipette was moved to the cell, showing green-fluorescent and barely attached to the cell membrane. Then, removing the positive pressure in the pipette and changing the holding potential to −80 mV, forming a tight seal between the cell membrane and the pipette. The seal resistance should be >=1GQ. The sealed membrane was excised to form a patch in an inside-out configuration. The recording started with a positive holding potential.

The patch was activated by 10 nM of capsaicin to activate TRPV1 channels fully. After full activation, 20 μM of drugs (cC) was applied to test for their ability of TRPV1 inhibition. The drug was applied in the desired volume of stock drug solution to 100 μL PBS for application. Drug was applied to achieve a concentration of 20 μM, with the DMSO solution below 0.2% v/v in the medium.

Traces were collected with the pClamp10 software suite, and the patch-clamp macroscopic current was recorded. The signals were amplified by Axon Axopatch 200B (Axon Instruments) and digitized by Axon Digidata 1550B (Axon Instruments). Trace was exported out in .abf file format and was analyze using pyabf and python to filter out traces and calculate the macroscopic current before and after drug application. The macroscopic current was summarized, and the drug potency was elucidated by comparing the changes in the macroscopic current.

### Mice

The mice used were 4-6 weeks old C57B/6J provided by APCF in HKUST. All animal procedures were approved by the University Committee on Research Practices of the Hong Kong University of Science and Technology (Ethics protocol number: 2016074).

### Capsaicin-Induced Pain-Sensation Assays

Capsaicin-induced pain-sensation assays in mice were performed where 10 μL of capsaicin (300 μM) or solvent control was injected into the top of the hind paw by using a fine (30-G) needle; capsaicin was dissolved in 0.3% ethanol and 3% (v/v) dimethyl sulfoxide (DMSO). The mice were wrapped gently in the investigator's hand and maintained on their back, with their snout pointing toward the small finger of the investigator; in this position, the mice remained unagitated during the injection. Mouse behavior was recorded using a digital camera (GoPro5).

The observation cage was made with the transparent large cage liner from APCF with bedding and food at the bottom. Mice were placed in the observation cage for 10 minutes at least 1 hour before the experiment was conducted to reduce the stress bought on by an unfamiliar environment. During the experiment, mice were captured by the left hand using the index finger and thumb to hold their neck. Then gradually pick up the mice and use the little finger to anchor its tail, such that its right hind paw will step on the ring finger or middle finger. Drugs were prepared in a 1.5 mL test tube. For the control group, 1 mM stock Capsaicin in DMSO is diluted to 100 μM using PBS. For the testing group, drugs originally dissolved in DMSO were diluted to the desired concentration in addition to capsaicin.

The injection was performed by shaking and spinning the drug-containing test tube for an even concentration of drug. A BD Ultra-fine 6 mm needle was used to suck out the 10 μL of drug from the needle. After removing excess air bubbles, 10 μL of the drug was injected into the captured mice's back of the right hind paw. The needle was removed slowly to inhibit blood and drug from leaking from the wound. After injection, mice were placed in the observation cage for 5 minutes to record the pain behavior. Video was filmed using a camera with the configuration of HD 30 fps. The environmental noise, light, and temperature are constant throughout the record. The mice's behavior was identified by experienced individuals who mapped pain behavior in seconds throughout the 5 minutes.

After the experiment, the mice were transferred to the original cage with warmth provided for their recovery. The mice were observed for one month.

### Statistics

All data are expressed as means±SEM; n denotes the number of independent biological replicates. Unless indicated otherwise, Student's two-tailed t-test was used for statistical analysis, and P<0.05 was considered statistically significant.

### EXEMPLARY EMBODIMENTS

Embodiment 1. A analgesic, comprising a hexacyclic xanthone in a vehicle.

Embodiment 2. The analgesic according to embodiment 1, wherein the hexacyclic xanthone is selected from chrexanthomycin A (cA), chrexanthomycin B (cB), chrexanthomycin C (cC), chrexanthomycin F (cF), or any combination thereof.

Embodiment 3. The analgesic according to embodiment 1, further comprising Capsazepine.

Embodiment 4. The analgesic according to embodiment 1, wherein the hexacyclic xanthone is at a concentration of about 0.75 to 20 μM.

Embodiment 5. The analgesic according to embodiment 1, wherein the vehicle is a solution comprising a solvent.

Embodiment 6. The analgesic according to embodiment 5, wherein the solvent comprises dimethylsulfoxide (DMSO).

Embodiment 7. A method of treating pain, comprising applying the analgesic according to embodiment 1 to a site of pain in a patient.

Embodiment 8. The method according to embodiment 7, wherein the hexacyclic xanthone is selected from chrexanthomycin A (cA), chrexanthomycin B (cB), chrexanthomycin C (cC), chrexanthomycin F (cF), or any combination thereof.

Embodiment 9. The method according to embodiment 7, wherein the vehicle is a solution comprising a solvent.

Embodiment 10. The method according to embodiment 9, wherein the solvent comprises DMSO.

Embodiment 11. The method according to embodiment 7, wherein applying comprises injection.

Embodiment 12. The method according to embodiment 7, wherein applying comprises topical administration.

All patents, patent applications, provisional applications, and publications cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

Brandt, M. R., Beyer, C. E., & Stahl, S. M. (2012). TRPV1 antagonists and chronic pain: Brandt beyond thermal perception. Pharmaceuticals, 5(2), 114-132.
Turk, D. C., & Melzack, R. (2011). The measurement of pain and the assessment of people experiencing pain.
Moran, M. M., & Szallasi, A. (2018). Targeting nociceptive transient receptor potential channels to treat chronic pain: current state of the field. British journal of pharmacology, 175(12), 2185-2203.
Yam, M. F., Loh, Y. C., Tan, C. S., Khadijah Adam, S., Abdul Manan, N., & Basir, R. (2018). General pathways of pain sensation and the major neurotransmitters involved in pain regulation. International journal of molecular sciences, 19(8), 2164.
Doly, S., Fischer, J., Salio, C., & Conrath, M. (2004). The vanilloid receptor-1 is expressed in rat spinal dorsal horn astrocytes. Neuroscience letters, 357(2), 123-126.
Cao, E., Liao, M., Cheng, Y., & Julius, D. (2013). TRPV1 structures in distinct conformations reveal activation mechanisms. Nature, 504(7478), 113-118.
Zhao, R., & Tsang, S. Y. (2017). Versatile roles of intracellularly located TRPV1 channel. Journal of Cellular Physiology, 232(8), 1957-1965.
Breese, N. M., George, A. C., Pauers, L. E., & Stucky, C. L. (2005). Peripheral inflammation selectively increases TRPV1 function in IB4-positive sensory neurons from adult mouse. Pain, 115(1-2), 37-49.
Ji, R. R., Samad, T. A., Jin, S. X., Schmoll, R., & Woolf, C. J. (2002). p38 MAPK activation by NGF in primary sensory neurons after inflammation increases TRPV1 levels and maintains heat hyperalgesia. Neuron, 36(1), 57-68.
Akbar, A., Yiangou, Y., Facer, P., Brydon, W. G., Walters, J. R., Anand, P., & Ghosh, S. (2010). Expression of the TRPV1 receptor differs in quiescent inflammatory bowel disease with or without abdominal pain. Gut, 59(6), 767-774.
Moran, M. M., & Szallasi, A. (2018). Targeting nociceptive transient receptor potential channels to treat chronic pain: current state of the field. British journal of pharmacology, 175(12), 2185-2203.
Brandt, M. R., Beyer, C. E., & Stahl, S. M. (2012). TRPV1 antagonists and chronic pain: beyond thermal perception. Pharmaceuticals, 5(2), 114-132.
Nilius, B., & Szallasi, A. (2014). Transient receptor potential channels as drug targets: from the science of basic research to the art of medicine. Pharmacological reviews, 66(3), 676-814.
Carroll, A. R., Copp, B. R., Davis, R. A., Keyzers, R. A., & Prinsep, M. R. (2019). Marine natural products. Natural product reports, 36(1), 122-173.
Haefner, B. (2003). Drugs from the deep: marine natural products as drug candidates. Drug discovery today, 8(12), 536-544.
Jiménez, C. (2018). Marine natural products in medicinal chemistry. ACS medicinal chemistry letters, 9(10), 959-961.
Molinski, T. F., Dalisay, D. S., Lievens, S. L., & Saludes, J. P. (2009). Drug development from marine natural products. Nature reviews Drug discovery, 8(1), 69-85.
Montaser, R., & Luesch, H. (2011). Marine natural products: a new wave of drugs. Future medicinal chemistry, 3(12), 1475-1489.
Mehbub, M. F., Lei, J., Franco, C., & Zhang, W. (2014). Marine sponge derived natural products between 2001 and 2010: trends and opportunities for discovery of bioactives. Marine drugs, 12(8), 4539-4577.
Khalifa, S. A., Elias, N., Farag, M. A., Chen, L., Saeed, A., Hegazy, M. E. F., . . . & El-Seedi, H. R. (2019). Marine natural products: A source of novel anticancer drugs. Marine drugs, 17(9), 491.
Lin, Z., Reilly, C. A., Antemano, R., Hughen, R. W., Marett, L., Concepcion, G. P., . . . & Schmidt, E. W. (2011). Nobilamides A-H, long-acting transient receptor potential vanilloid-1 (TRPV1) antagonists from mollusk-associated bacteria. Journal of medicinal chemistry, 54(11), 3746-3755.
Wu, Y., Yeh, F. L., Mao, F., & Chapman, E. R. (2009). Biophysical characterization of styryl dye-membrane interactions. Biophysical journal, 97(1), 101-109.
Nishikawa, S. (2011). Fluorescent AM1-43 and FM1-43 probes for dental sensory nerves and cells: Their labeling mechanisms and applications. Japanese Dental Science Review, 47(2), 150-156.
Meyers, J. R., MacDonald, R. B., Duggan, A., Lenzi, D., Standaert, D. G., Corwin, J. T., & Corey, D. P. (2003). Lighting up the senses: FM1-43 loading of sensory cells through nonselective ion channels. Journal of Neuroscience, 23(10), 4054-4065.
Gaffield, M. A., & Betz, W. J. (2006). Imaging synaptic vesicle exocytosis and endocytosis with FM dyes. Nature protocols, 1(6), 2916-2921.
Kornreich, B. G. (2007). The patch clamp technique: principles and technical considerations. Journal of Veterinary Cardiology, 9(1), 25-37.
Chen, C. C., Cang, C., Fenske, S., Butz, E., Chao, Y. K., Biel, M., . . . & Grimm, C. (2017). Patch-clamp technique to characterize ion channels in enlarged individual endolysosomes. Nature Protocols, 12(8), 1639-1658.
Phillips, E., Reeve, A., Bevan, S., & McIntyre, P. (2004). Identification of species-specific determinants of the action of the antagonist capsazepine and the agonist PPAHV on TRPV1. Journal of Biological Chemistry, 279(17), 17165-17172.
Abbas, M. A. (2020). Modulation of TRPV1 channel function by natural products in the treatment of pain. Chemico-biological interactions, 330, 109178.
Kym, P. R., Kort, M. E., & Hutchins, C. W. (2009). Analgesic potential of TRPV1 antagonists. Biochemical pharmacology, 78(3), 211-216.
Cheng, A., Liu, C., Ye, W., Huang, D., She, W., Liu, X., . . . & Qian, P. Y. (2022). Selective C9or f72 G-Quadruplex-Binding Small Molecules Ameliorate Pathological Signatures of ALS/FTD Models. Journal of Medicinal Chemistry, 65(19), 12825-12837.
Cao, X., Zhou, Z., Tian, Y., Liu, Z., Cheng, K. O., Chen, X., . . . & Huang, P. (2021). Opposing roles of E3 ligases TRIM23 and TRIM21 in regulation of ion channel ANO1 protein levels. Journal of Biological Chemistry, 296.

We claim:

1. A analgesic, comprising a hexacyclic xanthone in a vehicle.

2. The analgesic according to claim 1, wherein the hexacyclic xanthone is selected from chrexanthomycin A (cA), chrexanthomycin B (cB), chrexanthomycin C (cC), chrexanthomycin F (cF), or any combination thereof.

3. The analgesic according to claim 1, further comprising Capsazepine.

4. The analgesic according to claim 1, wherein the hexacyclic xanthone is at a concentration of about 0.75 to 20 μM.

5. The analgesic according to claim 1, wherein the vehicle is a solution comprising a solvent.

6. The analgesic according to claim 5, wherein the solvent comprises dimethylsulfoxide (DMSO).

7. A method of treating pain, comprising applying the analgesic according to claim 1 to a site of pain in a patient.

8. The method according to claim 7, wherein the hexacyclic xanthone is selected from chrexanthomycin A (cA), chrexanthomycin B (cB), chrexanthomycin C (cC), chrexanthomycin F (cF), or any combination thereof.

9. The method according to claim 7, wherein the vehicle is a solution comprising a solvent.

10. The method according to claim 9, wherein the solvent comprises DMSO.

11. The method according to claim 7, wherein applying comprises injection.

12. The method according to claim 7, wherein applying comprises topical administration.
